(19) United States
(12) Patent Application Publication
(10) Pub. No.: US 2025/0137935 A1
(43) Pub. Date: May 1, 2025

(54) METHOD FOR ASSESSING QUALITY OF AMINE-BASED SHALE INHIBITORS

(71) Applicant: SAUDI ARABIAN OIL COMPANY, Dhahran (SA)

(72) Inventors: Vikrant Wagle, Abqaiq (SA); Omprakash Pal, Dhahran (SA); Abdullah Al-Yami, Dhahran (SA); Sara Alkhalaf, Al Khobar (SA)

(73) Assignee: SAUDI ARABIAN OIL COMPANY, Dhahran (SA)

(21) Appl. No.: 18/495,188

(22) Filed: Oct. 26, 2023

(51) Int. Cl. G01N 21/82; G01N 31/02; G01N 33/28

(52) U.S. Cl. CPC G01N 21/82; G01N 31/02; G01N 33/2823

(57) ABSTRACT

Described is a method for assessing quality of an amine-based shale inhibitor in drilling fluid. A relationship between a concentration of the amine-based shale inhibitor in a drilling fluid and turbidity is established by preparing drilling fluid solutions with varying concentrations of the amine-based shale inhibitor, allowing the drilling fluid solutions to form precipitates, and analyzing turbidity of the drilling fluid solutions. A sample of a drilling fluid with an unknown concentration of amine-based shale inhibitor is obtained. Using the established relationship, a concentration of the amine-based shale inhibitor in the sample of the drilling fluid is estimated.

### BACKGROUND

Amine-based compounds are commonly used as shale inhibitors in water-based drilling fluids. Amine-based shale inhibitors are added in predetermined concentrations in water-based drilling fluids. Amine-based shale inhibitors prevent the swelling and disintegration of shales, thereby preventing problems during the drilling process, such as wellbore instability, viscosity build-up, high torque and drag, bit balling, stuck pipe, and wellbore caving. However, the shale inhibitors are generally consumed or lost to the formation during the drilling process. Therefore, it is important to maintain optimal amine concentrations in the drilling fluids for effective drilling.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method for assessing a quality of an amine-based shale inhibitor. A relationship between a concentration of an amine-based shale inhibitor in a drilling fluid and turbidity is established by preparing a plurality of drilling fluid solutions with a plurality of varying concentrations of the amine-based shale inhibitor, each drilling fluid solution having a distinct concentration of the amine-based shale inhibitor. The plurality of drilling fluid solutions is allowed to form precipitates, and turbidity of the plurality of drilling fluid solutions is analyzed. One or more samples of a drilling fluid with an unknown concentration of amine-based shale inhibitor is obtained. Using the established relationship, a concentration of the amine-based shale inhibitor in the one or more samples of the drilling fluid is estimated.

In another aspect, turbidity of the one or more samples of the drilling fluid is analyzed.

In another aspect, analyzing turbidity of the one or more samples of the drilling fluid comprises determining turbidity values for the one or more samples of the drilling fluid based on absorption intensities obtained from a turbidimeter.

In another aspect, the estimated concentration is correlated to a quality of the amine-based shale inhibitor in the one or more samples of drilling fluid.

In another aspect, the one or more samples of the drilling fluid are obtained prior to a drilling operation.

In another aspect, the one or more samples of the drilling fluid are obtained from a circulating drilling fluid during a drilling operation.

In another aspect, analyzing turbidity of the plurality of drilling solutions comprises determining turbidity values for the plurality of drilling fluid solutions based on absorption intensities obtained from a turbidimeter.

In another aspect, the established relationship is a calibration plot.

In another aspect, a plurality of water-based drilling fluid samples having a plurality of varying concentrations of the amine-based shale inhibitor are formulated.

In another aspect, preparing the plurality of drilling fluid solutions comprises collecting a mud filtrate from each water-based drilling fluid sample; and mixing an amount of each mud filtrate with an amount of an amine precipitating reagent.

In another aspect, the amine precipitating reagent is Reinecke's salt.

In another aspect, the plurality of varying concentrations of the amine-based shale inhibitor range from 0 pounds per barrel (ppb) to 10 ppb.

In another aspect, the plurality of varying concentrations of the amine-based shale inhibitor range from 1 ppb to 2 ppb.

In another aspect, the plurality of varying concentrations of the amine-based shale inhibitor range from 2 ppb to 4 ppb.

In another aspect, the plurality of varying concentrations of the amine-based shale inhibitor range from 4 ppb to 6 ppb.

In another aspect, the plurality of varying concentrations of the amine-based shale inhibitor range from 6 ppb to 8 ppb.

In another aspect, the plurality of varying concentrations of the amine-based shale inhibitor range from 8 ppb to 10 ppb.

Other aspects and advantages of the claimed subject matter will be apparent from the following description and the appended claims.

### BRIEF DESCRIPTION OF DRAWINGS

FIG. 1 illustrates a reaction of amine with Reinecke's salt according to one or more embodiments of the present disclosure.

FIG. 2 is a flow diagram illustrating a method for determining the concentration of amine-based shale inhibitor in water-based drilling fluids according to one or more embodiments of the present disclosure.

FIG. 3 illustrates a relationship between turbidity values measured and KLA-STOP™ concentrations in aqueous solutions according to one or more embodiments of the present disclosure.

FIG. 4 illustrates a calibration plot of a relationship between turbidity values and KLA-STOP™ concentrations according to one or more embodiments of the present disclosure.

FIG. 5 illustrates a computing system according to one or more embodiments of the present disclosure.

FIG. 6 illustrates a method for assessing quality of an amine-based shale inhibitor according to one or more embodiments of the present disclosure.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

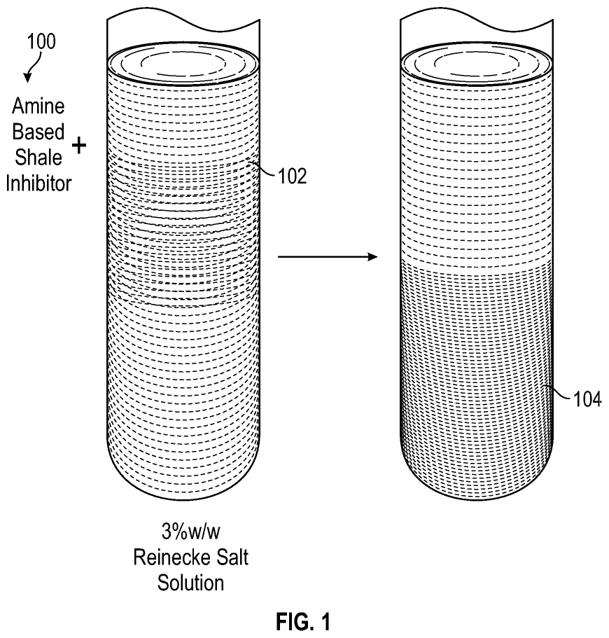

In the following description of FIGS. 1-6, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a passive soil gas sample system” includes reference to one or more of such systems.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

In one aspect, embodiments disclosed herein relate to a turbidimetric method for assessing quality of an amine-based shale inhibitor in a drilling fluid. The concentration of amine-based shale inhibitors in a drilling fluid, such as a water-based drilling fluid, may be determined by reacting a solution of an amine shale inhibitor with an amine precipitating reagent. In one embodiment, the amine precipitating reagent is Reinecke's salt. The resulting solution may be characterized by turbidimetry to estimate the amount of amine in the solution. The amount of amine in the solution may be used to characterize the quality of the amine shale inhibitor.

Reinecke's salt is a commercially available dark-red crystalline compound (having the formula: NH₄[Cr(NCS)₄(NH₃)₂]·H₂O) that is soluble in boiling water and ethanol. The salt may be used to precipitate primary, secondary, tertiary, and quaternary amines as their ammonium salts. In the method disclosed herein, varying concentrations of amine-based shale inhibitor 100 are reacted with a Reinecke's salt solution 102, as shown in FIG. 1. The reaction produces a white precipitate 104 in the solution. The precipitate 104 may be analyzed visually or with a turbidimeter. As can be appreciated by one skilled in the art, the method to assess the precipitate is not limited to the aforementioned techniques.

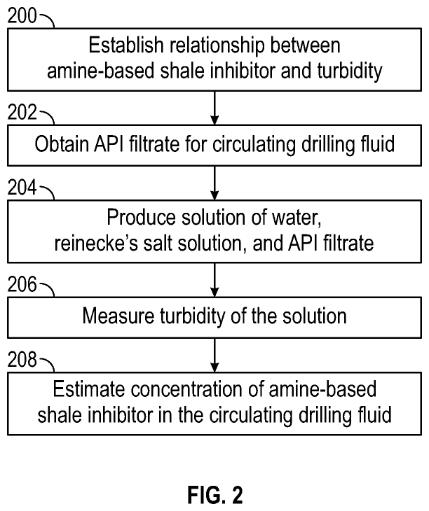

In one embodiment, a relationship between varying amine concentrations in drilling fluid mixed with Reinecke's salt solution and turbidimetry is determined. FIG. 2 is a flow diagram illustrating a method for determining the concentration of amine-based shale inhibitor in water-based drilling fluids in real-time conditions during drilling using turbidimetry. In block 200, a relationship between varying concentrations of amine-based shale inhibitor in the drilling fluid and turbidity is established in the form of a calibration plot. API low-temperature and low-pressure fluid loss measurements are performed on circulating drilling fluid to obtain the API filtrate for the circulating drilling fluid in block 202. In block 204, a solution including a mixture of water, Reinecke's salt solution, and API filtrate of the circulating drilling fluid is produced and kept static for a determined amount of time sufficient for precipitation. In block 206, the turbidity of the solution is measured using a standard turbidimeter. In block 208, the concentration of amine-based shale inhibitor is estimated from the calibration plot.

### EXAMPLES

The reaction of varying concentrations of amine-based shale inhibitor with Reinecke's salt was investigated. KLA-STOP™ polyamine shale inhibitor was used as the amine-based shale inhibitor. KLA-STOP™ is commercially available through SLB located at 509 W. Hensley Blvd., Bartlesville, OK 74003. As can be appreciated by one skilled in the art, other amine-based shale inhibitors may be used and evaluated other than KLA-STOP™.

### Example 1-Detection of Amine-Based Shale Inhibitor in Aqueous Solutions

The objective of the experiments was to establish a relationship between varying amine concentrations and the amount of precipitate formed using a turbidimetry method. Aqueous solutions of KLA-STOP™ were prepared at distinct concentrations, including 0.5 pounds per barrel (ppb), 1 ppb, 2 ppb, 3 ppb, 4 ppb, and 5 ppb. A Reinecke's salt indicator solution was prepared by dissolving approximately 3% weight by weight (w/w) of Reinecke's salt into deionized water and filtering the solution through a filter, such as a 0.45 micrometer (μm) filter.

The following procedure was followed during the investigation. First, aqueous solutions of KLA-STOP™, or another amine-based shale inhibitor, at different concentrations were prepared in a range of 0 pounds per barrel (ppb) to 5 ppb, such as 0.5 ppb, 1 ppb, 2 ppb, 3 ppb, 4 ppb, and 5 ppb. In some embodiments, the varying concentrations of KLA-STOP™ are selected from a range of 0 ppb to 1 ppb, 1 ppb to 2 ppb, 2 ppb to 3 ppb, 3 ppb to 4 ppb, or 4 ppb to 5 ppb. A solution of Reinecke's salt indicator solution was prepared by dissolving approximately 3% w/w (weight by weight) of Reinecke's salt into deionized water. The prepared solution was shaken to form a mixture and kept static for an amount of time, such as 25 minutes, then filtered through a 0.45 μm filter. The amount of precipitate formed was measured using a standard turbidimeter. A turbidimeter measures the cloudiness of a liquid sample by determining how much light can pass through the liquid. The greater the amount of amine present in the aqueous solution, the greater the amount of precipitate formed after reacting with Reinecke's salt.

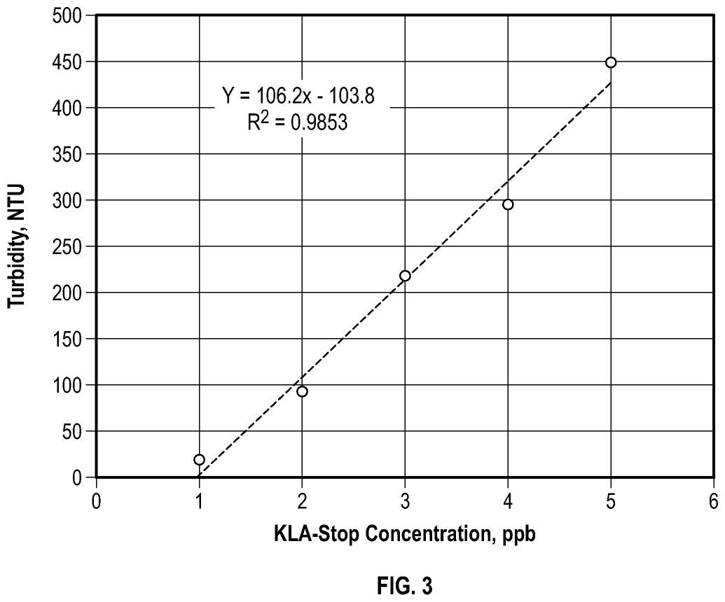

FIG. 3 depicts the relationship between turbidity values measured for six aqueous solutions and the concentration of KLA-STOP™ in the six aqueous solutions. The amount of precipitate formed correlated with the concentration of the amine (KLA-STOP™) from 0.5 ppb to 5 ppb in the aqueous solutions. In other words, the amount of precipitate increased with increasing concentrations of amine.

### Example 2-Detection of Amine-Based Shale Inhibitor in Water-Based Drilling Fluids

The reaction of KLA-STOP™ polyamine shale inhibitor in water-based drilling fluids with Reinecke's salt was investigated. The objective was to establish a relationship, such as in the form of a calibration plot, of different concentrations of KLA-STOP™ in the water-based drilling fluids and turbidity values obtained using a turbidimeter. The established relationship may then be utilized to assess the quality of an amine-based shale inhibitor in a drilling fluid circulating through a wellbore during a drilling operation.

The following procedure was followed to establish the relationship between varying KLA-STOP™ concentrations in water-based drilling fluids and turbidity. Various 75 pounds-force per cubic foot (pcf) water-based drilling fluids with a plurality of varying concentrations of KLA-STOP™ were formulated. In one or more embodiments, the plurality of varying concentrations of KLA-STOP™ range from 0 ppb to 10 ppb, such as 1 ppb to 2 ppb, 2 ppb to 4 ppb, 4 ppb to 6 ppb, 6 ppb to 8 ppb, and 8 ppb to 10 ppb. The water-based drilling fluids compositions are listed in Table 1.

TABLE 1

| | Mixing | | | | | | |
|---|---|---|---|---|---|---|---|
| Additives | time (min) | Fluid 1 | Fluid 2 | Fluid 3 | Fluid 4 | Fluid 5 | Fluid 6 |
| Water, ppb | | 308.5 | 306.7 | 304.9 | 303.9 | 303.1 | 301.3 |
| NaCl, ppb | 2 | 55.00 | 55.00 | 55.00 | 55.00 | 55.00 | 55.00 |
| XC polymer | 5 | 0.50 | 0.50 | 0.50 | 0.50 | 0.50 | 0.50 |
| PAC L, ppb | 5 | 2.00 | 2.00 | 2.00 | 2.00 | 2.00 | 2.00 |
| KLA-STOP ™ ppb | 5 | 2 | 3 | 6 | 7 | 8 | 10 |
| Caustic soda, ppb | 5 | 0.50 | 0.50 | 0.50 | 0.50 | 0.50 | 0.50 |
| Barite, ppb | 5 | 51.9 | 51.9 | 51.6 | 51.5 | 51.4 | 51.2 |

Following preparation of the drilling fluids, American Petroleum Institute (API) low-temperature and low-pressure fluid loss measurements were performed on all of the drilling fluids, and the corresponding API mud filtrates were collected. A Reinecke's salt indicator solution was prepared by dissolving approximately 3% w/w of Reinecke's salt reagent into deionized water and filtering the solution through a 0.45 μm filter. To a small vial or bottle, about 8 milliliters (ml) water, about 2 ml of 3% w/w Reinecke's salt solution, and about 2 ml of the API filtrate was added. The vial was shaken slightly for the ingredients to mix properly. The vial was kept static for approximately 25 min, and the precipitate was allowed to settle. The turbidity of the aqueous solution was measured using a standard turbidimeter.

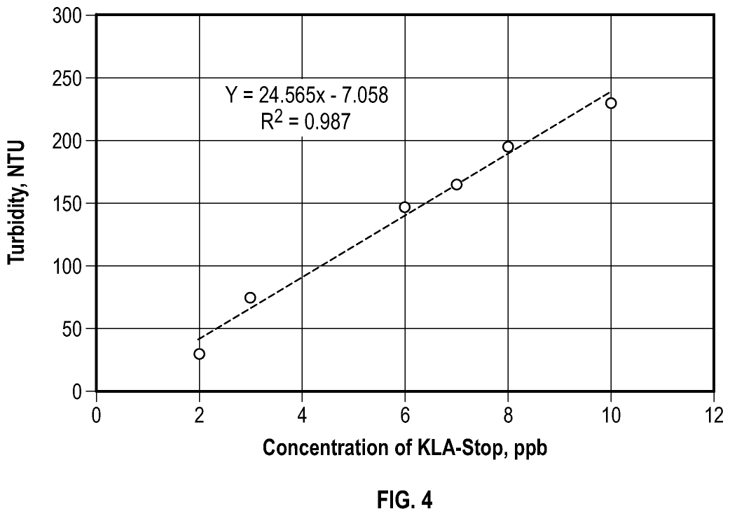

FIG. 4 is a plot of turbidity values for the six water-based drilling fluids formulated with varying concentrations of KLA-STOP™. As shown, turbidity values increase as the KLA-STOP™ concentration in the water-based drilling fluids increases. A correlation in the form of a calibration plot of different concentrations of KLA-STOP™ amine-based shale inhibitor versus turbidity values was, thus, established for the 75 pcf drilling fluid. Turbidity values were determined based on absorption intensities obtained from a standard turbidimeter.

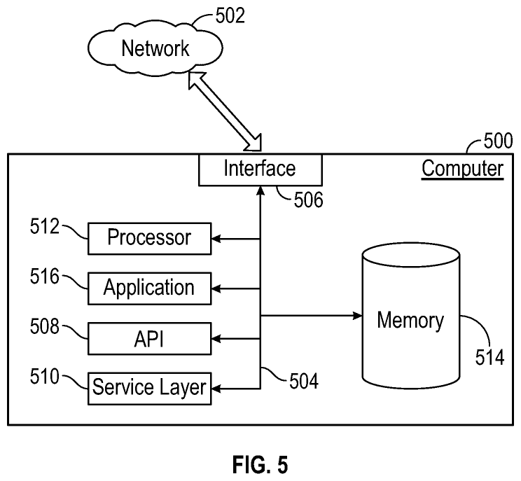

FIG. 5 depicts a block diagram of a computer 500 used to provide computational functionalities associated with described calculations, algorithms, methods, functions, processes, flows, and procedures as described in this disclosure, according to one or more embodiments. The illustrated computer 500 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 500 may include an input device, such as a keypad, keyboard, touch screen, or other device that can accept user information, and an output device that conveys information associated with the operation of the computer 500, including digital data, visual, or audio information (or a combination of information), or a GUI.

The computer 500 can serve in a role as a client, network component, a server, a database or other persistency, or any other component (or a combination of roles) of a computer system for performing the subject matter described in the instant disclosure. The illustrated computer 500 is communicably coupled with a network 502. In some implementations, one or more components of the computer 500 may be configured to operate within environments, including cloud-computing-based, local, global, or other environments (or a combination of environments).

At a high level, the computer 500 is an electronic computing device operable to receive, transmit, process, store, or manage data and information associated with the described subject matter. According to some implementations, the computer 500 may also include or be communicably coupled with an application server, e-mail server, web server, caching server, streaming data server, business intelligence (BI) server, or other server (or a combination of servers).

The computer 500 can receive requests over network 502 from a client application (for example, executing on another computer 500) and responding to the received requests by processing the said requests in an appropriate software application. In addition, requests may also be sent to the computer 500 from internal users (for example, from a command console or by other appropriate access method), external or third-parties, other automated applications, as well as any other appropriate entities, individuals, systems, or computers.

Each of the components of the computer 500 can communicate using a system bus 504. In some implementations, any or all of the components of the computer 500, both hardware or software (or a combination of hardware and software), may interface with each other or the interface 506 (or a combination of both) over the system bus 504 using an application programming interface (API) 508 or a service layer 510 (or a combination of the API 508 and service layer 510). The API 508 may include specifications for routines, data structures, and object classes. The API 508 may be either computer-language independent or dependent and refer to a complete interface, a single function, or even a set of APIs. The service layer 510 provides software services to the computer 500 or other components (whether or not illustrated) that are communicably coupled to the computer 500. The functionality of the computer 500 may be accessible for all service consumers using this service layer. Software services, such as those provided by the service layer 510, provide reusable, defined business functionalities through a defined interface. For example, the interface may be software written in JAVA, C++, or other suitable language providing data in extensible markup language (XML) format or another suitable format. While illustrated as an integrated component of the computer 500, alternative implementations may illustrate the API 508 or the service layer 510 as stand-alone components in relation to other components of the computer 500 or other components (whether or not illustrated) that are communicably coupled to the computer 500. Moreover, any or all parts of the API 508 or the service layer 510 may be implemented as child or sub-modules of another software module, enterprise application, or hardware module without departing from the scope of this disclosure.

The computer 500 includes an interface 506. Although illustrated as a single interface 506 in FIG. 5, two or more interfaces 506 may be used according to particular needs, desires, or particular implementations of the computer 500. The interface 506 is used by the computer 500 for communicating with other systems in a distributed environment that are connected to the network 502. Generally, the interface 506 includes logic encoded in software or hardware (or a combination of software and hardware) and operable to communicate with the network 502. More specifically, the interface 506 may include software supporting one or more communication protocols associated with communications such that the network 502 or interface's hardware is operable to communicate physical signals within and outside of the illustrated computer 500.

The computer 500 includes at least one computer processor 512. Although illustrated as a single computer processor 512 in FIG. 5, two or more processors may be used according to particular needs, desires, or particular implementations of the computer 500. Generally, the computer processor 512 executes instructions and manipulates data to perform the operations of the computer 500 and any algorithms, methods, functions, processes, flows, and procedures as described in the instant disclosure.

The computer 500 also includes a memory 514 that holds data for the computer 500 or other components (or a combination of both) that can be connected to the network 502. For example, memory 514 can be a database storing data consistent with this disclosure. Although illustrated as a single memory 514 in FIG. 5, two or more memories may be used according to particular needs, desires, or particular implementations of the computer 500 and the described functionality. While memory 514 is illustrated as an integral component of the computer 500, in alternative implementations, memory 514 can be external to the computer 500.

The application 516 is an algorithmic software engine providing functionality according to particular needs, desires, or particular implementations of the computer 500, particularly with respect to functionality described in this disclosure. For example, application 516 can serve as one or more components, modules, applications, etc. Further, although illustrated as a single application 516, the application 516 may be implemented as multiple applications 516 on the computer 500. In addition, although illustrated as integral to the computer 500, in alternative implementations, the application 516 can be external to the computer 500.

There may be any number of computers 500 associated with, or external to, a computer system containing computer 500, wherein each computer 500 communicates over network 502. Further, the term “client,” “user,” and other appropriate terminology may be used interchangeably as appropriate without departing from the scope of this disclosure. Moreover, this disclosure contemplates that many users may use one computer 500, or that one user may use multiple computers 500.

Formations that contain hydratable shale or mixed layers of reactive clay may lead to wellbore instability issues. The analytical method described herein tracks shale inhibitor concentration through quantitative measurements. Additionally, the method may be used for quality control of amine-based shale inhibitors. As a result, it is possible to achieve effective shale inhibition improving wellbore stability, maximizing drilling efficiency, and enhancing control over the wellbore.

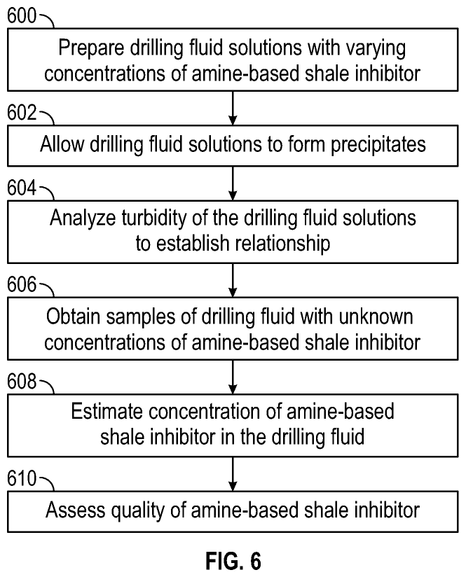

The method described above may be used to assess the quality of an amine-based shale inhibitor in a drilling fluid, as illustrated in FIG. 6. A relationship between a concentration of a standard amine-based inhibitor and turbidity is first established and may be visualized as a calibration plot. For the purposes of this disclosure, “standard” refers to an amine-based shale inhibitor having the highest purity that may be obtained. To establish the relationship, drilling fluid solutions with varying concentrations of the standard amine-based shale inhibitor are prepared in step 600, each drilling fluid solution having a distinct concentration of the standard amine-based shale inhibitor. After allowing the drilling fluid solutions to form precipitates in step 602, the turbidity of the drilling fluid solutions is analyzed in step 604. Then, one or more samples of a drilling fluid with an unknown concentration of amine-based shale inhibitor is obtained in step 606. In one or more embodiments, the one or more drilling fluid samples are obtained after the addition of the amine-based shale inhibitor but prior to any drilling operations. Alternatively, the one or more drilling fluid samples may be obtained from circulating drilling fluids during or after drilling operations have been performed. In one or more embodiments, samples of drilling fluid are obtained both before and after drilling operations to assess the concentration of amine-based shale inhibitor after drilling.

Using the relationship established between the standard amine-based inhibitor and turbidity, an active concentration of the amine-based shale inhibitor in the one or more samples of drilling fluid is estimated in step 608. The turbidity correlates to the amount of precipitate formed such that the greater the size, or amount, of the precipitate, the greater the turbidity and the higher the concentration of the amine-based shale inhibitor. A higher concentration of amine-based shale inhibitor in drilling fluid samples indicates a higher quality amine-based shale inhibitor, since shale inhibitors may be consumed or lost during the drilling process. Thus, the quality of the amine-based shale inhibitor in the drilling fluid is assessed in step 610. The calibration plot may be used to determine the quality of the shale inhibitor obtained from any vendor or supplier.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. A method for assessing a quality of an amine-based shale inhibitor, comprising:
establishing a relationship between a concentration of an amine-based shale inhibitor in a drilling fluid and turbidity, wherein establishing the relationship comprises:
preparing a plurality of drilling fluid solutions with a plurality of varying concentrations of the amine-based shale inhibitor, each drilling fluid solution having a distinct concentration of the amine-based shale inhibitor;
allowing the plurality of drilling fluid solutions to form precipitates;
analyzing turbidity of the plurality of drilling fluid solutions;
obtaining one or more samples of a drilling fluid with an unknown concentration of amine-based shale inhibitor; and
using the established relationship, estimating a concentration of the amine-based shale inhibitor in the one or more samples of the drilling fluid.

2. The method of claim 1, further comprising analyzing turbidity of the one or more samples of the drilling fluid.

3. The method of claim 2, wherein analyzing turbidity of the one or more samples of the drilling fluid comprises determining turbidity values for the one or more samples of the drilling fluid based on absorption intensities obtained from a turbidimeter.

4. The method of claim 1, wherein the estimated concentration is correlated to a quality of the amine-based shale inhibitor in the one or more samples of the drilling fluid.

5. The method of claim 1, wherein the one or more samples of the drilling fluid are obtained prior to a drilling operation.

6. The method of claim 1, wherein the one or more samples of the drilling fluid are obtained from a circulating drilling fluid during a drilling operation.

7. The method of claim 1, wherein a plurality of samples of the drilling fluid are obtained prior to a drilling operation and from a circulating drilling fluid during a drilling operation.

8. The method of claim 1, wherein analyzing turbidity of the plurality of drilling solutions comprises determining turbidity values for the plurality of drilling fluid solutions based on absorption intensities obtained from a turbidimeter.

9. The method of claim 1, wherein the established relationship is visualized as a calibration plot.

10. The method of claim 1, wherein preparing the plurality of drilling fluid solutions comprises formulating a plurality of water-based drilling fluid samples having a plurality of varying concentrations of the amine-based shale inhibitor.

11. The method of claim 10, wherein preparing the plurality of drilling fluid solutions further comprises:
collecting a mud filtrate from each water-based drilling fluid sample; and
mixing an amount of each mud filtrate with an amount of an amine precipitating reagent.

12. The method of claim 11, wherein the amine precipitating reagent is Reinecke's salt.

13. The method of claim 1, wherein the plurality of varying concentrations of the amine-based shale inhibitor range from 0 pounds per barrel (ppb) to 10 ppb.

14. The method of claim 13, wherein the plurality of varying concentrations of the amine-based shale inhibitor range from 1 ppb to 2 ppb.

15. The method of claim 13, wherein the plurality of varying concentrations of the amine-based shale inhibitor range from 2 ppb to 4 ppb.

16. The method of claim 13, wherein the plurality of varying concentrations of the amine-based shale inhibitor range from 4 ppb to 6 ppb.

17. The method of claim 13, wherein the plurality of varying concentrations of the amine-based shale inhibitor range from 6 ppb to 8 ppb.

18. The method of claim 13, wherein the plurality of varying concentrations of the amine-based shale inhibitor range from 8 ppb to 10 ppb.
